(19) United States
(12) Patent Application Publication
(10) Pub. No.: US 2024/0207511 A1
(43) Pub. Date: Jun. 27, 2024

(54) DEVICE FOR SUBCUTANEOUS DELIVERY OF A MEDICAMENT

(71) Applicant: Nuova Ompi S.r.l. Unipersonale, Padua (IT)

(72) Inventors: Marco Bellusci, Arcore (MB) (IT); Mattia Cattaneo, Limbiate (MB) (IT); Paolo Degan, Ranica (BG) (IT); Christian Riva, Cinisello Balsamo (MI) (IT); Nicola Bonserio, Giovinazzo (BA) (IT); Andrea Giacomozzi, Milano (MI) (IT)

(21) Appl. No.: 18/545,606

(22) Filed: Dec. 19, 2023

(30) Foreign Application Priority Data
Dec. 21, 2022 (IT) 102022000026286

(51) Int. Cl. A61M 5/145

(52) U.S. Cl. CPC A61M 5/14566; A61M 5/1454; A61M 2005/14573; A61M 2205/121

(57) ABSTRACT

A device for subcutaneous delivery of a medicament includes a dispensing module with a base wall applied to skin of a patient, an elongated seat arranged inside the dispensing module, the elongated seat including a rest portion, a cartridge arranged in the elongated seat and including a container holding the medicament, an elongated opening formed on the base wall at the elongated seat to allow the insertion of the cartridge into the elongated seat, and retaining members that interact with the cartridge and lock the cartridge inside the elongated seat . The device has an initial configuration where the retaining members do not act on the cartridge and the cartridge rests on the rest portion and is movable in the elongated seat along a longitudinal direction, and a final configuration where the retaining members interact with the cartridge and lock the cartridge in a predetermined position.

### CROSS REFERENCE TO RELATED APPLICATIONS

The present application claims priority to Italian Patent Application No. 102022000026286, filed Dec. 21, 2022, the disclosure of which is incorporated herein by reference in its entirety.

### TECHNICAL FIELD

The present disclosure relates to a device for subcutaneous delivery of a medicament. In particular, the present disclosure relates to a wearable type device configured to be applied on the body of a patient in order to allow the subcutaneous delivery of a predetermined dose of medicament.

### BACKGROUND

Wearable delivery devices can include medicament that is initially contained in a cartridge housed inside the device and is transferred to the body of the patient via a fluidic path.

The cartridge can include a cylindrical container, made of a plastic or glass material, a plunger slidable inside the container to push the medicament out of the container and a pierceable septum that guarantees the sterility of the container until the beginning of the delivery of the therapy. The fluidic path of wearable delivery devices includes a piercing needle to pierce the pierceable septum of the cartridge, an injection needle, and a flexible tube that puts the piercing needle in fluid communication with the injection needle. The container of the cartridge is kept closed by the pierceable septum until the delivery of the medicament is required. When such a delivery is required, the piercing needle pierces the pierceable septum and opens the fluidic path, allowing the medicament to reach the patient by initially passing through the piercing needle, then through the flexible tube and finally through the injection needle, due to the thrust exerted by the plunger on the medicament inside the container.

### SUMMARY OF THE DISCLOSURE

The present disclosure relates to devices for subcutaneous delivery of a medicament including a dispensing module, an elongated seat arranged inside the dispensing module, a cartridge arranged in the elongated seat, an elongated opening, and retaining members.

In some embodiments of devices for subcutaneous delivery, the cartridge is housed in a housing seat specifically provided in the device.

Devices for subcutaneous delivery of medicaments of the prior art adopt different solutions for inserting the cartridge into the housing seat.

For example, in the devices described in US 20220031940A1 and U.S. Pat. No. 8,157,769B2 the cartridge is inserted by the patient into the respective housing seat before using the device by sliding the cartridge along a direction parallel to or coincident with the extension direction of the housing seat.

Differently, in the device described in U.S. Pat. No. 10,251,995B2 the cartridge is first inserted by the patient into the housing seat along an insertion direction which is oblique to the direction along which a cartridge housing support located within the device extends. Subsequently, the cartridge is rotated so as to lay it on the housing support.

Still differently, in the device described in U.S. Pat. No. 10,307,529B2, the insertion of the cartridge into the housing seat by the patient takes place along a direction perpendicular to the extension direction of the housing seat.

It is advantageous to contain the cost of the devices for subcutaneous delivery of medicaments as much as possible, which can be achieved by speeding up and simplifying the procedure for inserting the cartridge into the respective housing seat.

In some implementations, to achieve this speeding up and simplification, the cartridge is inserted into the respective housing seat along a direction perpendicular to the longitudinal direction of the housing seat, such as described in U.S. Pat. No. 10,307,529B2. This insertion method can be suitable for being easily automated. However, in some instances, after having placed the cartridge in the housing seat, the cartridge can be locked in a predetermined position (hereinafter also referred to as nominal position), this position being suitable for ensuring the correct alignment of the cartridge with the piercing needle before and during the step of piercing the pierceable septum. Furthermore, in instances in which the cartridge is arranged in the housing seat during the mounting or assembly of the device, the correct alignment between cartridge and piercing needle must also be maintained during packaging, transport, storage and use of the device, and also following shocks, vibrations or falls of the device. In some implementations, the correct alignment between cartridge and piercing needle can be maintained even in those cases in which the device is subject to temperature variations during storage and transport, since such temperature variations may cause changes in the sizes of the components of the device and in the volumes of the medicaments.

In the device described in U.S. Pat. No. 10,307,529B2, the locking in position of the cartridge in the housing seat takes place due to the thrust exerted by a closing door of the housing seat on a locking element keyed in the centre of the container of the cartridge or at an end portion of the container of the cartridge. This thrust causes the locking element (and therefore the cartridge) to be pressed against a hydraulic driving unit provided inside the device on the opposite side to the closing door with respect to the cartridge, realizing a shape coupling between grooves and protrusions provided in the locking element and grooves and protrusions provided in the hydraulic driving unit. Such a shape coupling prevents any movement of the cartridge inside the housing seat.

The solution described in U.S. Pat. No. 10,307,529B2 for locking the cartridge in position inside the housing seat is cumbersome. For example, this solution requires keying the abovementioned locking element on the container of the cartridge. This inevitably causes an increase in the thickness of the device, with the consequence of making it uncomfortable when it is worn by the patient. A further drawback correlated to the provision of the locking element is that it complicates the mechanical structure of the cartridge and makes it compatible only with a specific device.

Furthermore, the provision of the locking element requires the implementation of an additional process step which could cause some drawbacks. For example, in those cases where the locking element is mounted on the cartridge by the cartridge manufacturer, it is necessary to verify that such a mounting is compatible with the subsequent procedures of sterilization and filling of the cartridge. On the other hand, in those cases where the locking element is mounted on the cartridge after having filled the cartridge with the medicament, it is necessary to verify that this mounting is compatible with the presence of the medicament inside the cartridge, since, for example, not all medicaments can be subjected to temperature variations or exposed to light.

In the present disclosure, a device can allow a cartridge to be inserted into the housing seat along a direction perpendicular to the longitudinal direction of the housing seat, so as to speed up and simplify the procedure for inserting the cartridge into the housing seat and consequently contain the production cost and sale cost of the device, and in which, however, the locking in position of the cartridge inside the housing seat is achieved by a less cumbersome and constructively simpler solution than the one described in U.S. Pat. No. 10,307,529B2, so as to contain the thickness of the device and not suffer the drawbacks described above.

In some instances, a simple and effective way to achieve the above aim is to provide at first for the cartridge to simply rest inside the housing seat without worrying about the desired alignment between cartridge and piercing needle being achieved and, subsequently, to interact with the cartridge along the longitudinal direction of the cartridge and of the housing seat to achieve the desired alignment between cartridge and piercing needle.

In some aspects, the present disclosure therefore relates to a device for subcutaneous delivery of a medicament, comprising:
a dispensing module comprising a base wall having a substantially flat outer surface configured to be applied to the skin of a patient;
an elongated seat arranged inside the dispensing module and extending along a longitudinal direction, the elongated seat comprising at least one rest portion;
a cartridge arranged in the elongated seat and comprising a container containing (e.g., holding) the medicament;
an elongated opening formed on the base wall at the elongated seat and configured to allow the insertion of the cartridge into the elongated seat along an insertion direction orthogonal to outer surface of the base wall;
retaining members configured to interact with the cartridge along a retaining direction parallel to or coincident with the longitudinal direction and to lock the cartridge inside the elongated seat in a predetermined position;
wherein the device has an initial configuration in which the retaining members do not act on the cartridge along the retaining direction and the cartridge rests on the at least one rest portion and is movable in the elongated seat along the longitudinal direction, and a final configuration in which the retaining members interact with the cartridge along the retaining direction and lock the cartridge in the predetermined position.

These, and other aspects, can include one or more of the following features. When the device is in the final configuration, the cartridge does not rest on the at least one rest portion. The retaining members can include a support element arranged at a first end portion of the elongated seat and a pushing element that, when the device is in the initial configuration, is arranged at a second end portion of the elongated seat, the pushing element being movable towards the support element to bring the device from the initial configuration to the final configuration. The container can include an end portion proximate to the support element, and the support element can include an abutment surface that, when the device is in the initial configuration, is in a position distal from the end portion and that, when the device is in the final configuration, is in abutment against the end portion. The cartridge can include a plunger sealingly arranged inside the container, and the pushing element can include a piston configured to exert a thrust on the plunger, where before reaching the final configuration, the thrust of the piston on the plunger causes a displacement of the cartridge towards the support element, and after reaching the final configuration, the thrust of the piston on the plunger causes the sliding of the plunger inside the container and the outflow of the medicament from the container through a piercing needle. The support element can include a rest element configured to support the end portion of the container when the device is in the initial configuration. The container can include a main portion and a neck having a reduced section, the neck being interposed between the main portion and the end portion, and when the device is in the initial configuration the neck rests on the rest element and the main portion rests on the at least one rest portion. When the device is in the final configuration, the neck does not rest on the rest element. The device can include a closing door configured to close the elongated opening. The closing door can be transparent. The closing door can be welded to the dispensing module. The closing door can include, on a surface of the closing door facing the elongated seat, a perimeter rib protruding towards the elongated seat and having a shape complementary to that of the profile of the cartridge. The perimeter rib does not contact the cartridge when the device is in the initial configuration and in the final configuration. The closing door can include, on the surface thereof facing the elongated seat, a plurality of reinforcing ribbings configured to reinforce the perimeter rib and extending from an outer side surface of the perimeter rib. The dispensing module can include an interface wall arranged on the opposite side to the base wall with respect to the cartridge, and can include an inspection window, where the cartridge is interposed between the inspection window and the closing door of the elongated opening.

In the device of the present disclosure, the cartridge can be inserted in the respective seat along a direction perpendicular to the longitudinal direction of the seat itself. This makes it possible to speed up and simplify the procedure for inserting the cartridge into the housing seat, to the benefit of the production cost and sale cost of the device. The locking of the cartridge in the desired position inside the housing seat takes place instead due to provision of retaining members which, by acting on the cartridge along the longitudinal direction of the cartridge and of the housing seat, do not cause an increase in the thickness of the device.

Furthermore, by not providing for the mounting of any additional element on the cartridge and, consequently, by not requiring any additional process steps, the device of the present disclosure is constructively simple and free of the drawbacks discussed above with reference to the prior art.

In certain aspects, a device for subcutaneous delivery of a medicament comprises retaining members of the type described above, and comprises a plunger slidably mounted inside the container, a piston having a rod and a disc which is configured to push the plunger, and a helical spring associated with (for example, connected to) the rod of the piston.

Certain features of the abovementioned devices are recited below. Each of these features can be provided individually or in combination with the others.

In some embodiments, when the device is in the final configuration the cartridge does not rest on the at least one rest portion. The locking in position of the cartridge inside the elongated seat when the cartridge has reached its nominal position therefore takes place solely due to an action exerted on the cartridge along the longitudinal direction of the elongated seat, i.e. without envisaging any interaction with the side surface of the cartridge.

In some embodiments, the rest portion has a cradle shape. This expedient allows both an easy positioning of the cartridge in the respective seat before the cartridge is locked in position, and an effective support of the cartridge when it moves inside the elongated seat before reaching its nominal position.

In some embodiments, the retaining members comprise a support element arranged at a first end portion of the elongated seat.

In some embodiments, the support element supports the cartridge when the device is in the initial configuration and when the device is in the final configuration.

In some embodiments, the retaining members comprise a pushing element that, when the device is in the initial configuration, is arranged at a second end portion of the elongated seat.

In some embodiments, the pushing element is movable towards the support element to bring the device from the initial configuration to the final configuration. When the device is in the final configuration the cartridge has reached its nominal position and remains locked in this position by the action exerted simultaneously by the support element and by the pushing element at the opposite end portions of the cartridge.

In some embodiments, the rest portion is arranged close to the second end portion of the elongated seat.

In some embodiments, the container of the cartridge comprises an end portion proximate to the support element.

In some embodiments, the support element comprises a rest element configured to support the end portion of the container when the device is in the initial configuration. This rest element cooperates with the rest portion provided in the elongated seat to effectively support the cartridge in the elongated seat at the opposite end portions thereof.

In some embodiments, the rest element has a cradle shape. As already mentioned with reference to the rest portion, this expedient allows both an easy positioning of the cartridge in the elongated seat before the cartridge is locked in position, and an effective support of the cartridge when it moves inside the elongated seat before the device reaches the final configuration.

In some embodiments, the support element comprises an abutment surface that, when the device is in the initial configuration, is in a position distal from the end portion and that, when the device is in the final configuration, is in abutment against the end portion. The thrust exerted by the pushing element on the cartridge thus brings the cartridge into abutment against the abovementioned abutment surface, reaching its nominal position. When the device is in the final configuration the cartridge is locked in this nominal position by the pushing action (e.g., force) exerted on the cartridge by the pushing element and by the abutment of the cartridge on the abutment surface provided on the rest element.

In some embodiments, the cartridge comprises a plunger sealingly arranged inside the container.

In some embodiments, the pushing element comprises a piston configured to exert a thrust on the plunger.

In some embodiments, before reaching the final configuration, the thrust of the piston on the plunger causes a displacement of the cartridge towards the support element.

In some embodiments, after reaching the final configuration the thrust of the piston on the plunger causes the sliding of the plunger inside the container and the outflow of the medicament from the container through a piercing needle.

Therefore, both the shift of the device from the initial configuration to the final configuration (and therefore the positioning of the cartridge in its nominal position) and the delivery of the medicament from the container towards the patient is a consequence of the pushing action, or force, exerted by the piston on the plunger. This pushing action at first causes the displacement of the cartridge into the elongated seat until the cartridge is locked in position inside the elongated seat, without any sliding of the plunger inside the container, and only later it causes the plunger to slide inside the container and the medicament to be delivered towards the patient. The locking of the cartridge in its nominal position therefore does not require further elements than those typically provided within the device to allow its normal use, with consequent containment of the overall dimensions of the device and simplification of the production and assembly operations.

In some embodiments, the container comprises a main portion and a neck having a reduced section interposed between the main portion and the end portion.

In some embodiments, when the device is in the initial configuration the neck rests on the rest element.

In some embodiments, when the device is in the initial configuration the main portion rests on the at least one rest portion.

In some embodiments, when the device is in the final configuration the neck does not rest on the rest element.

In some embodiments, the device comprises a closing door configured to close the elongated opening.

In some embodiments, the closing door is transparent. This expedient allows both to see the cartridge and the medication contained therein, since the cartridges are also typically made of a transparent material, and to automate the procedure for controlling the correct positioning of the cartridge inside the device during the shift from the initial configuration of the device to the final configuration of the device (also referred to herein as pre-setting step), thus reducing the production times of the device. In fact, the pre-setting step provides for the continuous monitoring, through an optical inspection system, of the relative position of the plunger and piston during the movement of the piston and the interruption of the movement of the piston when the cartridge has reached its nominal position.

In some embodiments, the closing door is welded to the dispensing module, so as to make the device tamper-proof.

In certain embodiments, the closing door is welded to the dispensing module by laser welding. This type of fixing is possible right because the closing door is transparent.

In some embodiments, the closing door comprises, on a surface thereof facing the elongated seat, a perimeter rib protruding towards the elongated seat.

In some embodiments, the perimeter rib has a shape complementary to that of the profile of the cartridge.

In some embodiments, the perimeter rib has a continuous profile.

In some embodiments, the perimeter rib does not contact the cartridge when the device is in the initial configuration.

In some embodiments, the perimeter rib does not contact the cartridge when the device is in the final configuration. In this way, the cartridge is free to move within the elongated seat during the pre-setting step. At the same time, the perimeter rib acts as a rest surface for the cartridge in the event of shocks and/or falls of the device, ensures that the cartridge is not damaged during the packaging and transport steps, and prevents unwanted excessive displacements of the cartridge from its nominal position.

In some embodiments, the closing door comprises, on the surface thereof facing the elongated seat, a plurality of reinforcing ribbings configured to reinforce the perimeter rib.

In some embodiments, the reinforcing ribbings extend from an outer side surface of the perimeter rib, so as not to hinder the view of the cartridge from outside the device.

In some embodiments, the dispensing module comprises an interface wall arranged on the opposite side to the base wall with respect to the cartridge.

In some embodiments, the device comprises a control module reversibly coupled to the dispensing module and configured to house at least one control unit and a battery to power the control unit.

In some embodiments, the control module comprises an interface surface facing the interface wall.

In some embodiments, the interface wall comprises an inspection window that allows the inspection of the medicament present inside the cartridge before starting the delivery of the medicament, and in particular before mutually coupling the dispensing module and the control module. In fact, it is advisable that the dispensing module is coupled to the control module only after having verified, by visual inspection, that the medicament present inside the cartridge does not have an altered appearance. It is also advisable to prevent the user from any possibility of access to the inspection window after the delivery device is coupled to the body of the patient and before the end of the delivery of the therapy. The inspection window is in fact accessible only before coupling the dispensing module and the control module together. In some instances, in order to access to the inspection window after having coupled the two modules, the two modules can be decoupled, which is an operation that can be done at the end of the delivery of the therapy.

In some embodiments, the cartridge is interposed between the inspection window and the closing door of the elongated opening. In this way, the light entering the dispensing module through the transparent closing door can reach the inspection window, facilitating the visual inspection of the medicament through the inspection window. The presence of the cartridge between the inspection window and the closing door on the other hand does not prevent the abovementioned light to reach the inspection window since the cartridge is made of a transparent material (and therefore it is permeable to light) to allow the medicament contained therein to be seen and the pre-setting step to be carried out.

In some embodiments, the elongated seat is arranged close to a continuous side wall of the dispensing module.

In some embodiments, the elongated seat is delimited, along the insertion direction, by the closing door of the elongated opening and by a bottom surface arranged on the opposite side to the elongated opening with respect to the cartridge.

In some embodiments, the elongated seat is delimited, along a direction orthogonal to the insertion direction and to the longitudinal direction, by the continuous side wall of the dispensing module.

In some embodiments, the elongated seat is delimited, along a direction orthogonal to the insertion direction and to the longitudinal direction, by at least one inner side wall arranged on the opposite side to the continuous side wall with respect to the cartridge. This inner side wall has the sole purpose of avoiding any unwanted interactions between the cartridge and the functional components specifically provided in the device to allow the delivery of the medicament to the patient. Therefore, the abovementioned inner side wall does not perform any function during the positioning of the cartridge inside the elongated seat and while reaching the nominal position of the cartridge.

In some examples, the elongated seat is delimited by walls and components within the device that are used for the operation of the device, possibly with the exception of the abovementioned inner side wall, with consequent containment of the overall dimensions of the device and simplification of the production and assembly operations.

In some embodiments, the device comprises a helical spring associated with the rod of the piston. This helical spring acts as a flexible rod of the piston and allows the piston and consequently the cartridge to advance during the pre-setting step and the plunger to advance inside the container of the cartridge during the step of delivery of the medicament.

In some embodiments, the helical spring comprises a plurality of first turns having a first inner diameter and, at a first free end of the helical spring, a plurality of second turns associated with the rod of the piston and having an inner diameter smaller than that of the first turns. The latter expedient makes it possible to facilitate and improve the coupling between the helical spring and the piston. The piston in turn prevents a direct interaction of the helical spring with the plunger, avoiding possible damages to the plunger. In addition, the piston allows the thrust generated by the advancement of the helical spring to be distributed over the entire surface of the plunger, thus allowing an easy advancement of the plunger inside the container of the cartridge. Moreover, the piston guarantees the seal inside the container and the correct coupling with the helical spring after the mounting of the device and before the pre-setting step. After this step, in fact, the thrust exerted by the helical spring would in any case tend to guarantee the correct mutual coupling between the helical spring and the piston, regardless of the correct coupling between the second turns of the helical spring and the rod of the piston.

In some embodiments, some of the first turns are rotationally coupled to an inner threading of a nut. The first turns thus act as a threaded fitting for the inner threading of the nut. When the nut is rotated, the helical spring moves with respect to the nut and pushes the piston against the plunger, pushing the cartridge when the device is in the initial configuration and the medicament after the device has reached the final configuration.

The choice of providing for the second turns an inner diameter smaller than that of the first turns allows to prevent the coupling between the helical spring and the piston from blocking the rotation of the nut because of the increase in volume of the helical spring at the piston.

In some embodiments, the nut comprises a circular outer profile.

In some embodiments, the outer profile is a toothed profile. The rotation of the nut is thus driven by a gear connected to a motor which is configured to activate the device.

In some embodiments, the rod of the piston has an outer diameter smaller than the inner diameter of the second turns.

In some embodiments, the rod of the piston comprises, on an outer side surface thereof, a plurality of longitudinal ribbings coupled by interference to the second turns.

In some embodiments, the ribbings are circumferentially equally spaced from each other. In this way the force exerted by the second turns on the rod of the piston is distributed substantially evenly along the entire outer surface of the rod of the piston.

In some embodiments, the ribbings have a length lower than that of the rod of the piston.

In some embodiments, each ribbing has a thickness lower than its length. The ribbings are therefore thin, so that they can deform when coupled by interference with the second turns of the helical spring. The second turns will in turn also deform when coupled by interference to the ribbings of the rod of the piston. However, this deformation does not involve also the first turns, which must in fact maintain their original shape to allow the helical spring to advance correctly inside the nut.

In some embodiments, the device comprises an anti-rotation member arranged at a second free end of the helical spring opposite the first free end. In this way, the helical spring does not rotate on itself due to the rotation of the nut. A rotation on itself of the helical spring may cause the displacement of the piston to be uncontrollable cause risk for the helical spring to rotate on itself without pushing the piston. In certain instances, the anti-rotation member can provide for a precise control of the displacement of the piston, including for a precise relationship between the number of turns of the nut and the displacement of the piston, in order to be able to deliver the correct dose of medicament.

In some embodiments, the anti-rotation member comprises a main body slidably connected to an inner surface of the base wall and a pin extending cantilevered from the main body and associated with the second free end of the helical spring.

In some embodiments, the main body has a height such that when the second free end of the helical spring is associated with the pin there are no portions of the helical spring that are at a height greater than that of the main body, so as to contain the thickness of the device.

In some embodiments, the pin has an outer diameter smaller than the inner diameter of the first turns.

In some embodiments, the main body comprises a recess slidably coupled to a track which is integral with the base wall of the dispensing module. The coupling between the recess of the main body and the track of the dispensing module allows the main body, and therefore the helical spring, to only be able to slide with respect to the dispensing module. The track in particular defines a path along which the main body, and thus the helical spring, slides when the nut rotates.

In some embodiments, the second free end of the helical spring comprises some of the first turns.

In some embodiments, the first turns of the helical spring have a predetermined winding pitch and the pin comprises an outer threading coupled to the abovementioned first turns and having a threading pitch equal to the winding pitch.

In some embodiments, the turns of the helical spring have a predetermined winding direction and the outer threading of the pin has a threading direction consistent with the winding direction.

In this description the threading direction is consistent with the winding direction of the turns of the helical spring if a rotation of the helical spring in the winding direction involves a screwing of the turns of the helical spring on the pin.

The anti-rotation mechanism described above does not require substantial structural changes to the helical spring, with consequent reduction in the production cost and purchase cost of the device, nor does it require special machining on the helical spring, thus avoiding the risk of introducing unwanted residual stresses due to such machining into the helical spring.

### BRIEF DESCRIPTION OF THE DRAWINGS

Further features and advantages of the present disclosure will become clearer from the following detailed description of preferred embodiments thereof, made with reference to the accompanying drawings and given for indicative and non-limiting purpose. In such drawings:

FIG. 1 is a perspective view of a device according to the present disclosure;

FIG. 2 is a perspective view of a module of the device of FIG. 1;

FIG. 3 is a perspective view of another module of the device of FIG. 1;

FIG. 4 is a view from above of the module of FIG. 2 without a cover;

FIG. 5 is an exploded perspective view from below of the module of FIG. 2;

FIG. 6 is an enlargement of a portion of FIG. 5 from a different point of view;

FIG. 7 is an enlargement of a detail of FIG. 6;

FIG. 8 is an enlargement of the portion of FIG. 7 from a different point of view;

FIG. 9 is a perspective view of another portion of FIG. 5 from a different point of view;

FIG. 10 is an enlargement of a part of the module of FIG. 2 in an operating configuration thereof;

FIG. 11 is a cross-sectional view of a detail of the module of FIG. 2;

FIG. 12 is a perspective top view of the module of FIG. 2 without the cover and some components;

FIG. 13 is a perspective sectional view of a portion of FIG. 12;

FIG. 14 is an enlargement of a component shown in FIG. 12;

FIG. 15 is a perspective sectional view of the portion of FIG. 13 in another operating configuration;

FIG. 16 is a side sectional view of another portion of FIG. 12;

FIG. 17 is an enlarged perspective view of a further portion of FIG. 12 without another component.

### DETAILED DESCRIPTION

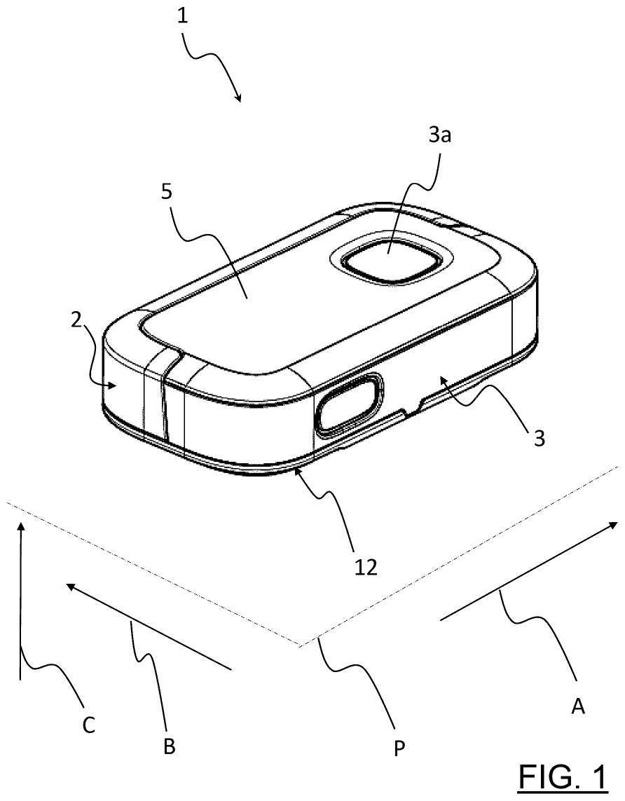

In FIG. 1, an example wearable type device for the subcutaneous delivery of a medicament in accordance with the present disclosure is indicated with 1. The application of the example device 1 on the body of a patient is carried out by a user, for example a doctor or a nurse or the patient.

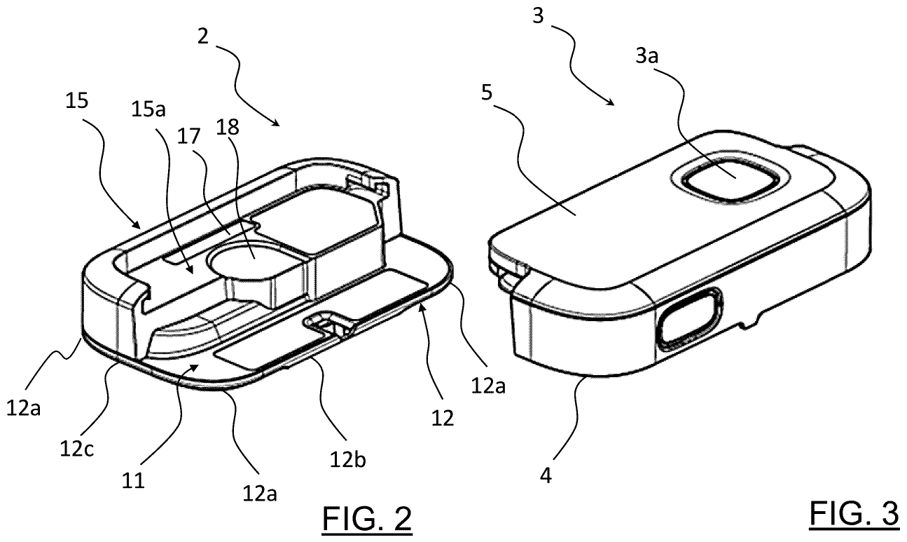

The example device 1 comprises a dispensing module 2, shown in FIG. 2, and a control module 3, shown in FIG. 3.

The example device 1 is obtained by mutually coupling the dispensing module 2 and the control module 3. This coupling is reversible, i.e. the two modules 2 and 3 after having been coupled can be decoupled. The control module 3 can be in a colour and/or material different from that of the dispensing module 2, so as to help the user to distinguish the two modules 2 and 3 during the coupling.

One or more gaskets can be interposed between the dispensing module 2 and the control module 3 in order to ensure the tightness of the mutual coupling.

In some instances, the dispensing module 2 is disposable, while the control module 3 is reusable. In other words, the dispensing module 2 is configured to be applied on the body of the patient only once and for a certain period of time to deliver the medicament, completely or in part, in one or more subsequent injections, even temporally spaced apart from each other. Differently, the control module 3 can be used several times, by coupling it from time to time with a new dispensing module 2.

As shown in FIG. 3, the control module 3 comprises a body 4 and a cover 5 associated with (e.g., connected to) the body 4, such as by ultrasonic welding. Ultrasonic welding can be preferable to laser welding, for example, because laser welding may require the use of transparent materials that are not preferred for the control module 3 since it can be customized with different colours and/or finishes.

In some implementations, a housing compartment is defined between the body 4 and the cover 5 and can contain a control unit configured to control the delivery of the medicament from the dispensing module 2, a motor that drives in rotation a driving magnetic rotor and a power supply battery configured to power the control unit and the motor.

The control module 3 further comprises an activation button 3a, by pressing which the patient operates the device 1.

In some implementations, such as shown in FIG. 2, the dispensing module 2 comprises a main body 11 and an intermediate cover 15 associated with (e.g., connected to) the main body 11 and interposed between the main body 11 and the body 4 of the control module 3. In some examples, the intermediate cover 15 is welded to the main body 11 by laser welding. Laser welding can be preferable to an ultrasonic welding, for example, because the assembly of the dispensing module 2 takes place with all the members described below already mounted on the main body 11 and the use of an ultrasonic welding can risk accidentally activating these members during the execution of the welding.

As shown in FIGS. 1 and 2, the main body 11 comprises a base wall 12 configured to come into contact with the body of the patient, for example, by interposition of an adhesive patch. In some implementations, the base wall 12 has a substantially flat outer surface defining a rest plane P to the skin of the patient. The base wall 12 can be made of a rigid material, such as a plastic material.

The base wall 12 has a substantially rectangular shape, with rounded corners 12a. The base wall 12 has two long sides 12b oriented along a first direction A parallel to the plane P and two short sides 12c oriented along a second direction B parallel to the plane P and orthogonal to the first direction A (FIGS. 1 and 2).

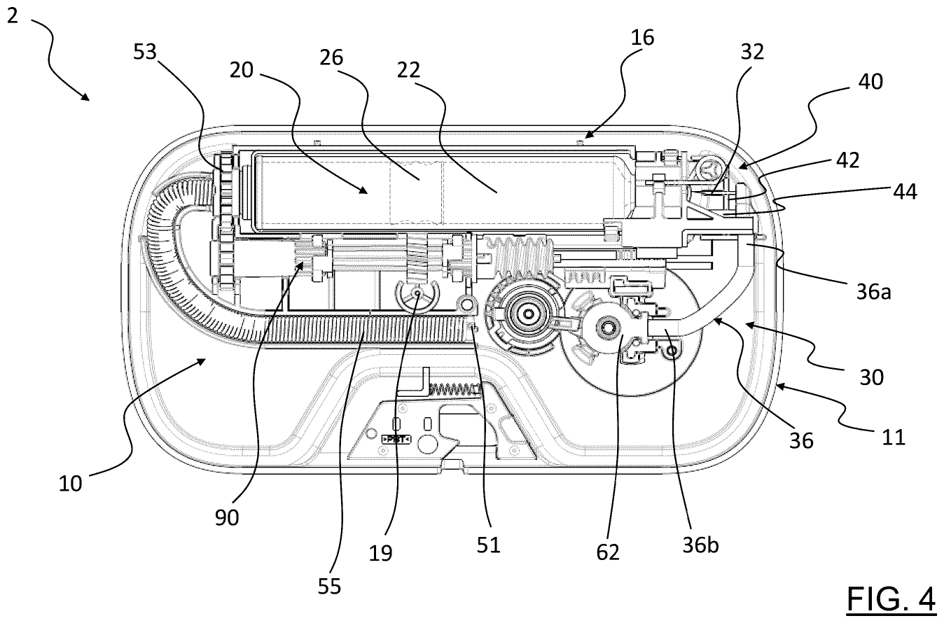

A housing compartment 10 is defined between the main body 11 and the intermediate cover 15 (FIG. 4).

The housing compartment 10 is configured to house a plurality of components, including a cartridge 20. As discussed above, the dispensing module 2 can be disposable and may not provide for the possibility of refilling or replacing the cartridge 20. It is therefore configured to be disposed of after removal of the device 1 from the body of the patient.

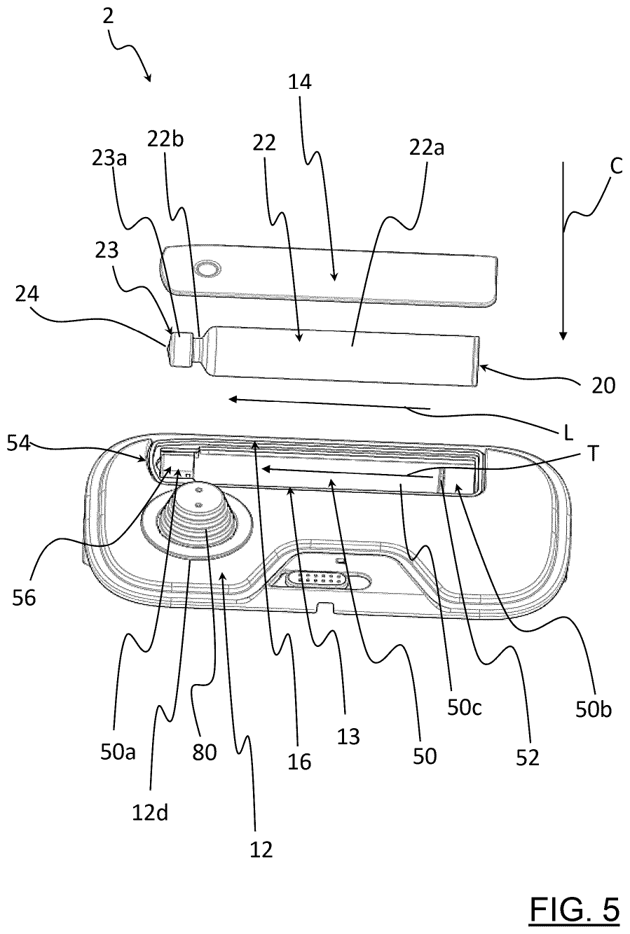

As shown in FIGS. 4 and 5, the cartridge 20 has an elongated shape and comprises a substantially cylindrical container 22, made of a plastic or glass material and containing (e.g., holding) the medicament to be delivered to the patient. A pierceable septum 24 that closes the container 22 and guarantees the sterility thereof until the device 1 is used is provided at one end portion 23 of the container 22.

The container 22 comprises a main portion 22a and a neck 22b having a reduced section interposed between the main portion 22a and the end portion 23. The pierceable septum 24 is retained by an aluminium ferrule 23a coupled to the end portion 23. A plunger 26 (shown in FIG. 4) is sealingly arranged within the container 22 and is slidable in the container 22 to deliver the medicament.

As shown in FIGS. 4 and 5, the cartridge 20 is housed with play in a housing seat 50, also referred to herein as “elongated seat”, specifically provided in the housing compartment 10 and having an elongated shape.

As shown in FIG. 5, an elongated opening 13 is formed in the base wall 12 at the elongated seat 50 to allow the insertion of the cartridge 20 into the elongated seat 50 during the assembly of the dispensing module 2.

The elongated opening 13 has a substantially rectangular shape and is sufficiently wide to allow the insertion of the cartridge 20 into the elongated seat 50 along an insertion direction C that is substantially orthogonal to the plane P of the base wall 12 (FIGS. 1 and 5), without therefore requiring rotations of the cartridge 20.

The elongated seat 50 has, in a plan view thereof, a substantially rectangular shape and extends along a longitudinal direction L which is substantially parallel to the first direction A.

The elongated seat 50 is arranged close to a continuous side wall 16 of the dispensing module 2. The side wall 16 defines a side wall of the elongated seat 50.

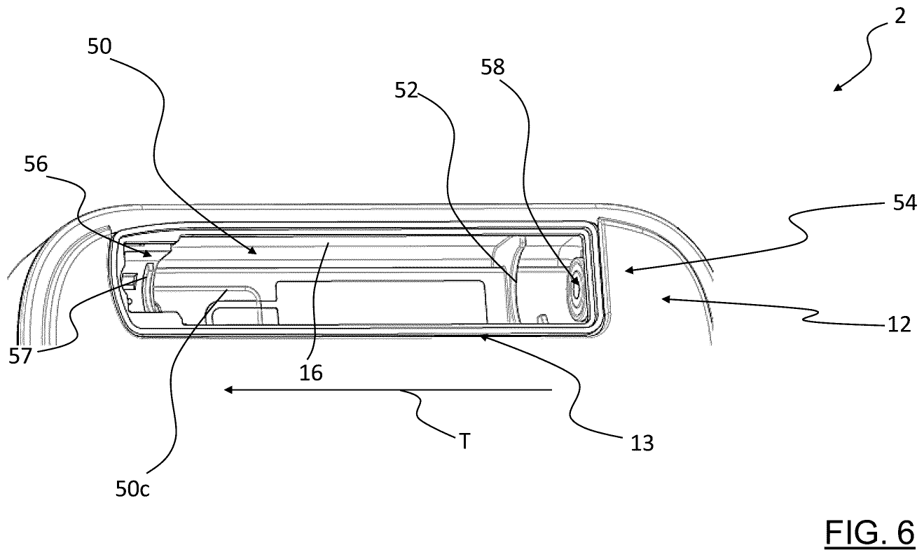

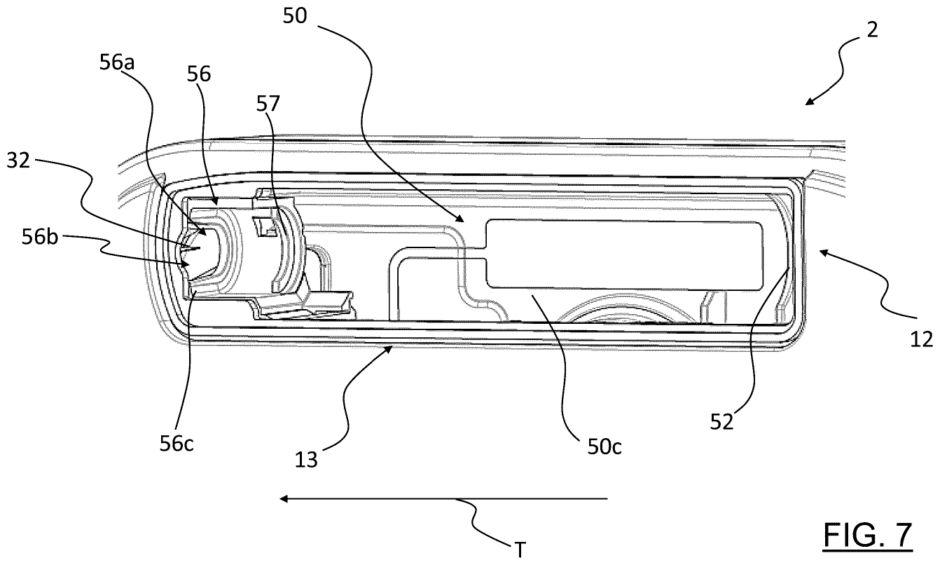

The elongated seat 50 is delimited, along the insertion direction C of the cartridge 20, by a closing door 14 described below and by a bottom surface 50c (FIGS. 6 and 7) arranged on the opposite side to the elongated opening 13 with respect to the cartridge 20.

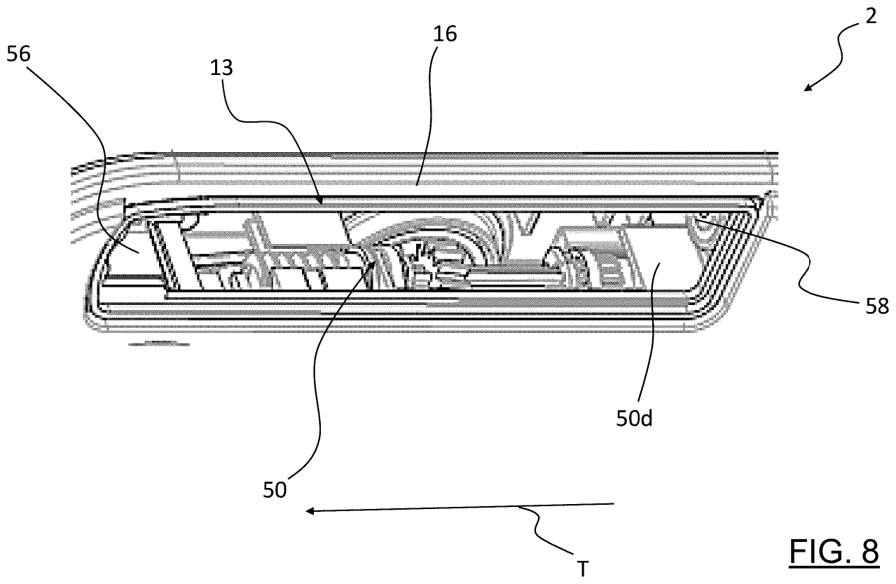

In some implementations, the elongated seat 50 is delimited, along a direction orthogonal to the insertion direction C and to the longitudinal direction L, by the continuous side wall 16 of the dispensing module 2 and, in some instances, by an inner side wall 50d (FIG. 8) arranged on the opposite side to the continuous side wall 16 with respect to the cartridge 20. The inner side wall 50d has the purpose of avoiding any possible interference between the cartridge 20 and the various functional members of the device 1 that are provided in the dispensing module 2. The inner side wall 50d is thus only a safety wall and does not define/guide the positioning of the cartridge 20 in the elongated seat 50. Multiple inner side walls or no inner side walls can also be foreseen.

As shown in FIG. 5, the elongated seat 50 comprises, close to an end portion 50b thereof, a rest portion 52 configured to support the main portion 22a of the container 22 of the cartridge 20 when the cartridge 20 is inserted into the elongated seat 50. This rest portion 52 has a cradle shape.

As shown in FIG. 4, the housing compartment 10 houses a plurality of components defining a fluidic path 30 configured to be travelled by the medicament during the subcutaneous delivery of the medicament, thus allowing the transfer of the medicament from the cartridge 20 to the body of the patient.

In the present description and in the subsequent claims, the term “fluidic path” is used to indicate any element or assembly of elements that is configured to be connected, at a first end thereof, to the cartridge housed inside the delivery device and comprising, at an end thereof opposite to the abovementioned first end, an injection needle intended to be inserted into the body of the patient to allow the transfer of the medicament from the cartridge to the body of the patient.

The fluidic path 30 comprises a piercing needle 32 configured to pierce the pierceable septum 24, an injection needle configured to be inserted into the body of the patient, and a flexible tube 36 that puts the abovementioned piercing needle 32 and injection needle in fluid communication.

In particular, the flexible tube 36 has an end portion 36a connected to a support member 42 that supports the piercing needle 32 and an opposite end portion 36b connected to a support member 62 that supports the injection needle.

The support member 42 is slidably mounted on a sliding guide 44 provided in the main body 11. The support member 42 is movable between a first operating position in which the piercing needle 32 is spaced apart from the pierceable septum 24 (as shown for example in FIG. 4) and the fluidic path 30 is closed, and a second operating position in which the piercing needle 32 has pierced the pierceable septum 24 and extends inside the container 22, thereby opening the fluidic path 30.

As shown in FIGS. 5-7, 10, and 11, the housing compartment 10 further houses a plurality of components defining retaining members 54 which are configured to retain the cartridge 20 in a nominal position inside the elongated seat 50 during the operation of the device 1. This nominal position is the one suitable for allowing the piercing needle 32 to penetrate inside the container 22 of the cartridge 20 reaching the depth which is necessary to allow the medicament to exit from the cartridge 20.

In particular, the retaining members 54, when acting on the cartridge 20, interact with the cartridge 20 along a retaining direction T parallel to or coincident with the longitudinal direction L.

The retaining members 54 comprise a support element 56 and a pushing element 58 arranged on the opposite side to the support element 56 with respect to the cartridge 20.

The support element 56 is arranged at an end portion 50a of the elongated seat 50 opposite to the end portion 50b and has a longitudinal through opening 56a facing towards the elongated opening 13 and configured to allow the insertion of the cartridge 20 in the elongated seat 50 orthogonally to the plane P through a mere translational movement. The support element 56 is shaped to accommodate with play the end portion 23 of the container 22, so as to compensate for the mounting tolerances of the ferrule 23a of the cartridge 20.

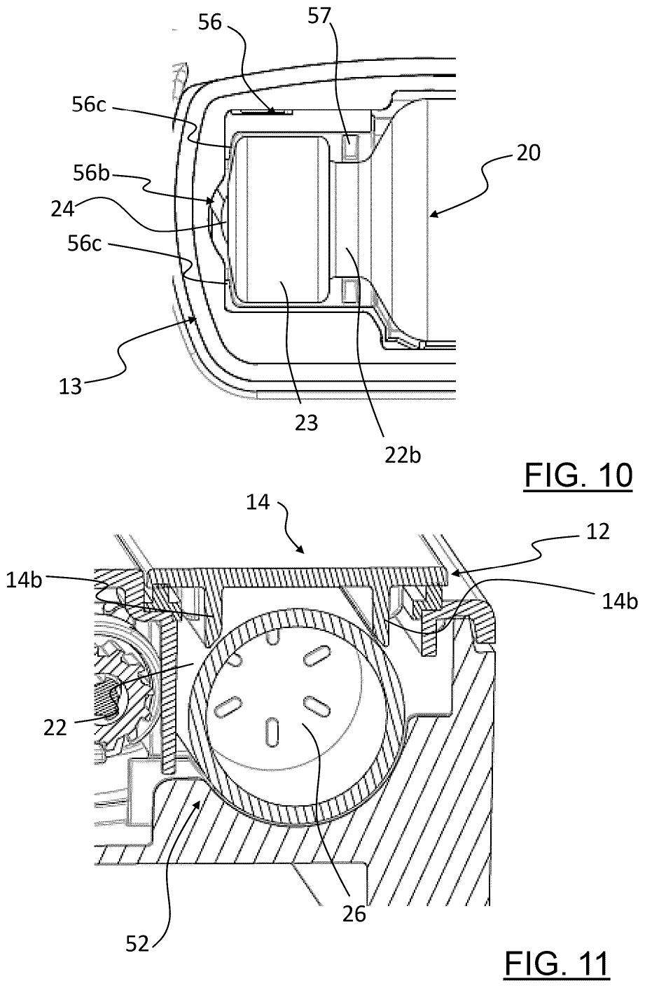

As shown in FIGS. 7 and 10, the support element 56 comprises an axial abutment surface 56c against which the end portion 23 of the cartridge 20 is configured to abut when the cartridge 20 is retained in position in the housing seat 50 by the retaining members 54. The abutment surface 56c has a substantially semi-annular shape and is arranged around a substantially semi-circular axial through opening 56b of the support element 56 which is configured to allow the passage of the piercing needle 32 (FIGS. 7 and 10).

The support element 56 also comprises a rest element 57 configured to support the neck 22b of the container 22 of the cartridge 20 when the cartridge 20 is inserted into the elongated seat 50.

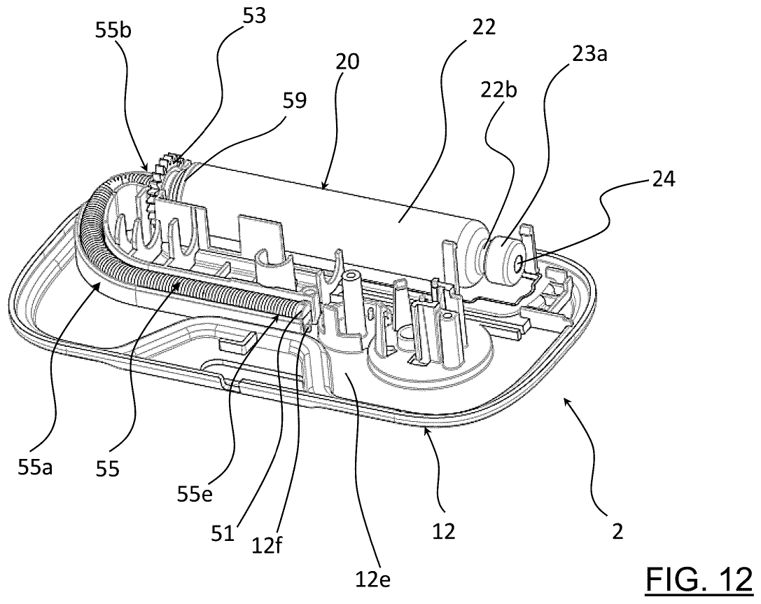

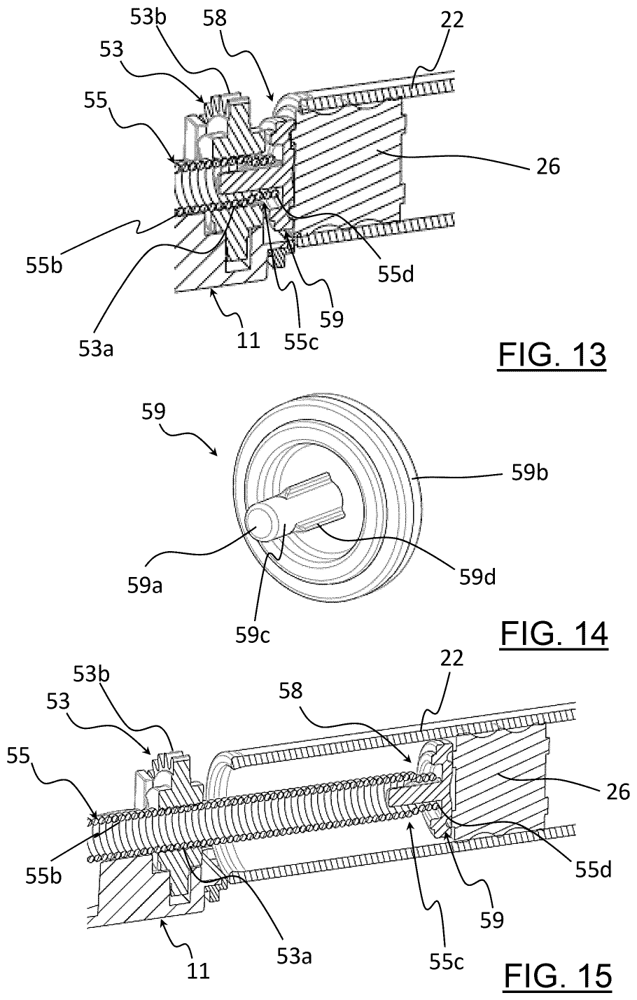

As shown in FIGS. 12-15, at first the pushing element 58 is arranged at the end portion 50b of the elongated seat 50 opposite to the end portion 50a.

The pushing element 58 is movable towards the support element 56 to allow the device 1 to shift from an initial configuration thereof to a final configuration thereof.

The pushing element 58 comprises a piston 59 comprising a rod 59a and a disc 59b extending substantially orthogonally to the rod 59a and configured to exert a thrust on the plunger 26.

A plurality of longitudinal ribbings 59d are formed on an outer side surface 59c of the rod 59a of the piston 59. Such ribbings 59d are circumferentially equally spaced from each other. They have a length lower than that of the rod 59a and a thickness lower than their length.

In the initial configuration of the device 1, the support element 56 and the piston 59 do not act on the cartridge 20 along the retaining direction T. In this initial configuration, the end portion 23 of the container 22 is in a position distal from the abutment surface 56c, the neck 22b of the container 22 rests on the rest element 57 and the main portion 22a of the container 22 rests on the rest portion 52. The cartridge 20 is movable in the elongated seat 50 along the longitudinal direction L.

In the final configuration of the device 1, the support element 56 and the piston 59 interact with the cartridge 20 along the retaining direction T and lock it inside the elongated seat 50 in the nominal position. In this final configuration, the end portion 23 of the container 22 is in abutment against the abutment surface 56c (FIG. 10) due to the thrust exerted by the disc 59b of the piston 59 on the plunger 26 and the cartridge 20 does not rest on the rest portion 52 and on the rest element 57.

After having reached the final configuration of the device 1, the thrust of the piston 59 on the plunger 26 causes the plunger 26 to slide inside the container 22 and the medicament to exit the container 22 through the piercing needle 32.

As shown in FIGS. 5, 6, 9 and 11, the device 1 comprises a closing door 14 configured to close the elongated opening 13. The closing door 14 has a substantially rectangular shape and is made of a transparent plastic material.

During the assembly of the dispensing module 2, after the cartridge 20 has been inserted into the elongated seat 50 through the elongated opening 13, the latter is closed by welding, for example, by laser welding, the closing door 14 to the dispensing module 2, so as to prevent tampering of the medicament contained in the cartridge 20.

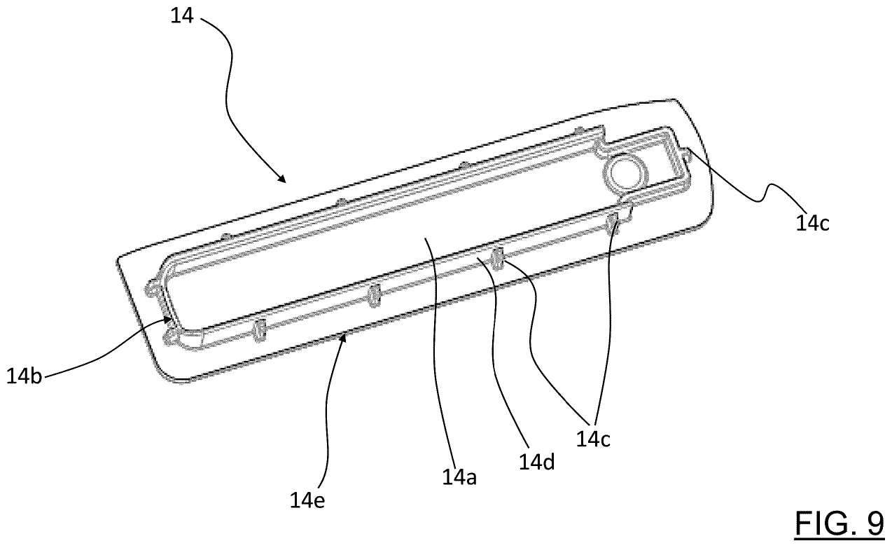

The closing door 14 comprises, on a substantially flat inner surface 14a thereof facing the elongated seat 50, a perimeter rib 14b protruding towards the elongated seat 50 (FIGS. 9 and 11) and a plurality of reinforcing ribbings 14c extending from an outer side surface 14d of the perimeter rib 14b.

The perimeter rib 14b has a continuous and closed profile. It has a shape which is substantially complementary to that of the profile of the cartridge 20.

The perimeter rib 14b is positioned close to a perimeter edge 14e of the closing door 14 so as not to obstruct the view of the cartridge 20 and the passage of light through the closing door 14.

When the device 1 is in the initial configuration and in the final configuration, the perimeter rib 14b does not contact the cartridge 20.

The perimeter rib 14b defines a continuous rest surface for the cartridge 20 in the event of shocks or falls of the device 1.

As shown in FIG. 2, the intermediate cover 15 of the dispensing module 2 comprises an interface wall 15a arranged on the opposite side to the base wall 12 with respect to the cartridge 20 and facing the control module 3 when the dispensing module 2 is coupled to the control module 3.

The interface wall 15a comprises an inspection window 17 configured to allow the visual inspection of the cartridge 20 when the control module 3 is decoupled from the dispensing module 2. Vice versa, when the control module 3 is coupled to the dispensing module 2, the inspection window 17 is covered by the control module 3.

The inspection window 17 is substantially parallel to the base wall 12.

The cartridge 20 is interposed between the inspection window 17 and the closing door 14 of the elongated opening 13.

The interface wall 15a further comprises a circular portion 18. The circular portion 18 is configured to allow the magnetic driving rotor of the control module 3 to rotatably actuate by magnetic coupling a magnetic rotor 19 provided in the dispensing module 2 at the circular portion 18 when the control module 3 is coupled to the dispensing module 2.

The delivery of the therapy comprises the controlled movement of the plunger 26 inside the container 22 towards the pierceable septum 24 due to the thrust exerted by the piston 59 when the device 1 is in the final configuration.

The piston 59 is moved by a helical spring 55 associated with the rod 59a of the piston 59 and extending inside the housing compartment 10 along a curved path 55a (FIG. 12).

The helical spring 55 is coupled to an inner threading 53a of a nut 53 comprising a toothed circular outer profile 53b.

The nut 53 is rotatably supported outside the elongated seat 50 close to the end portion 50b of the elongated seat 50.

The nut 53 is driven to rotate by a medicament dispensing mechanism 90 housed in the housing compartment 10 (FIGS. 4, 12, 13 and 15). The rotation of the nut 53 causes the helical spring 55 to translate, which pushes the piston 59, thus displacing the plunger 26 (FIG. 15).

The helical spring 55 comprises a plurality of first turns 55b having a predetermined winding pitch, a predetermined winding direction and a first inner diameter and, at a first free end 55c of the helical spring 55, a plurality of second turns 55d associated with the rod 59a of the piston 59 and having a winding pitch and a winding direction equal to those of the first turns 55b and an inner diameter smaller than that of the first turns 55b and greater than the outer diameter of the rod 59a of the piston 59.

The first turns 55b are coupled in rotation to the inner threading 53a of the nut 53, while the second turns 55d are coupled by interference to the ribbings 59d of the rod 59a of the piston 59.

The helical spring 55 thus acts as a flexible shaft of the piston 59 and as a threaded fitting, so as to realize a coupling of the screw-nut screw type with the nut 53.

In order to prevent the rotation of the helical spring 55 on itself when the nut 53 is rotated, the dispensing module 2 comprises, at a second free end 55e of the helical spring 55 opposite to the first free end 55c, an anti-rotation member 51.

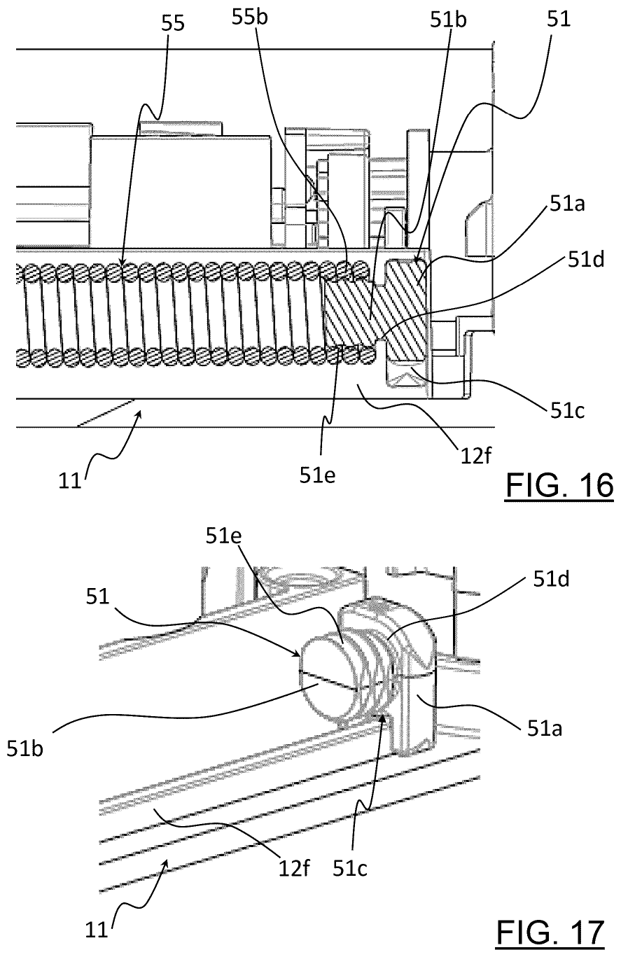

As shown in FIGS. 16 and 17, this anti-rotation member 51 comprises a main body 51a and a pin 51b extending cantilevered from the main body 51a substantially orthogonally to the latter.

A circumferential recess 51d is interposed between the main body 51a and the pin 51b.

The pin 51b has an outer diameter smaller than the inner diameter of the first turns 55b and is associated with the second free end 55e of the helical spring 55.

In particular, the pin 51b comprises an outer threading 51e coupled to the first turns 55b and having a threading pitch equal to the winding pitch of the first turns 55b and a threading direction consistent with the winding direction of the helical spring 55.

The main body 51a is configured such that, when the helical spring 55 is screwed onto the pin 51b, the total height of the anti-rotation member 51 with respect to the plane P is lower than the maximum height of the helical spring 55 with respect to the plane P.

The main body 51a comprises a recess 51c slidably coupled to a track 12f which is integral with an inner surface 12e of the base wall 12 of the dispensing module 2. The helical spring 55 is not depicted in the partial perspective view of the example device of FIG. 17 to depict the sliding coupling between the recess 51c and the track 12f.

The assembly of the dispensing module 2 comprises inserting the cartridge 20 into the elongated seat 50, closing the elongated opening 13 with the closing door 14 and welding the closing door 14 onto the base wall 12 of the main body 11 of the dispensing module 2.

At this point, the pre-setting step is carried out on the dispensing module 2 in order to bring the cartridge 20 into the nominal position and the device 1 into the final configuration.

The pre-setting step comprises, in particular:
driving a magnetic rotor contained in the dispensing module 2 to drive in rotation the nut 53 and therefore to translate the helical spring 55 so as to bring the disc 59b of the piston 59 into abutment against the plunger 26;
during the movement of the piston 59, monitoring the relative position of the plunger 26 and of the disc 59b of the piston 59 by the optical inspection system;
interrupting the movement of the piston 59 when the end portion 23 of the container 22 abuts against the abutment surface 56c.

During the pre-setting step, the cartridge 20 is thus free to move within the elongated seat 50. The rest element 57 of the support element 56, the rest portion 52 and the perimeter rib 14b of the closing door 14 allow enough play to the cartridge 20 to settle during the pre-setting step but at the same time they prevent the cartridge 20 from taking unsuitable positions inside the elongated seat 50.

At the end of the pre-setting step the device 1 is in the final configuration and the cartridge 20 is kept in abutment against the abutment surface 56c by the axial pressure exerted by the disc 59b of the piston 59. The cartridge 20 is thus kept in the nominal position by purely axial forces.

The rest element 57, the rest portion 52 and the perimeter rib 14b perform the function of safety containment structures. Therefore, under normal conditions, the cartridge 20 does not rest on such containment structures, but if following shocks, vibrations and/or falls of the device 1 the axial pressure were not able to keep the cartridge 20 in its nominal position, it cannot in any case translate until losing its nominal position, because the play allowed by the containment structures is so limited that any misalignment of the cartridge 20 cannot significantly move the end portion 23 of the container 22 away from its correct position.

What is claimed is:

1. A device for subcutaneous delivery of a medicament, comprising:
a dispensing module comprising a base wall having a substantially flat outer surface configured to be applied to skin of a patient;
an elongated seat arranged inside the dispensing module and extending along a longitudinal direction, the elongated seat comprising at least one rest portion;
a cartridge arranged in the elongated seat and comprising a container holding the medicament;
an elongated opening formed on the base wall at the elongated seat and configured to allow an insertion of the cartridge into the elongated seat along an insertion direction that is orthogonal to the outer surface of the base wall; and
retaining members configured to interact with the cartridge along a retaining direction and to lock the cartridge inside the elongated seat in a predetermined position, the retaining direction being parallel to or coincident with the longitudinal direction;
wherein:
the device has an initial configuration in which the retaining members do not act on the cartridge along the retaining direction and the cartridge rests on the at least one rest portion and is movable in the elongated seat along the longitudinal direction; and
the device has a final configuration in which the retaining members interact with the cartridge along the retaining direction and lock the cartridge in the predetermined position.

2. The device of claim 1, wherein, when the device is in the final configuration, the cartridge does not rest on the at least one rest portion.

3. The device of claim 2, wherein the retaining members comprise:
a support element arranged at a first end portion of the elongated seat; and
a pushing element that, when the device is in the initial configuration, is arranged at a second end portion of the elongated seat, the pushing element being movable towards the support element to transition the device from the initial configuration to the final configuration.

4. The device according to claim 3, wherein:
the container comprises an end portion proximate to the support element; and
the support element comprises an abutment surface that, when the device is in the initial configuration, is in a position distal from the end portion and that, when the device is in the final configuration, is in abutment against the end portion.

5. The device of claim 4, wherein:
the cartridge comprises a plunger sealingly arranged inside the container; and
the pushing element comprises a piston configured to exert a thrust on the plunger, wherein the thrust of the piston on the plunger is configured to causes a displacement of the cartridge towards the support element before the device reaches the final configuration, and wherein the thrust of the piston on the plunger is configured to cause a sliding of the plunger inside the container and an outflow of the medicament from the container through a piercing needle after the device reaches the final configuration.

6. The device of claim 3, wherein the support element comprises a rest element configured to support the end portion of the container when the device is in the initial configuration.

7. The device of claim 6, wherein the container comprises a main portion and a neck having a reduced section, the neck being interposed between the main portion and the end portion, wherein, when the device is in the initial configuration, the neck rests on the rest element and the main portion rests on the at least one rest portion.

8. The device of claim 7, wherein, when the device is in the final configuration, the neck does not rest on the rest element.

9. The device of claim 1, comprising a closing door configured to close the elongated opening.

10. The device of claim 9, wherein the closing door is transparent.

11. The device of claim 10, wherein the closing door is welded to the dispensing module.

12. The device of claim 11, wherein the closing door comprises, on a surface of the closing door facing the elongated seat, a perimeter rib protruding towards the elongated seat and having a shape complementary to a profile of the cartridge.

13. The device of claim 12, wherein the perimeter rib does not contact the cartridge when the device is in the initial configuration and in the final configuration.

14. The device of claim 12, wherein the closing door comprises, a plurality of reinforcing ribbings on the surface of the closing door and facing the elongated seat, the plurality of reinforcing ribs being configured to reinforce the perimeter rib and extending from an outer side surface of the perimeter rib.

15. The device of claim 14, wherein:
the dispensing module comprises an interface wall arranged on a side to the base wall opposite the cartridge, the dispensing module comprising an inspection window; and
the cartridge is interposed between the inspection window and the closing door of the elongated opening.

16. The device of claim 9, wherein:
the dispensing module comprises an interface wall arranged on a side to the base wall opposite the cartridge and the dispensing module comprising an inspection window; and
the cartridge is interposed between the inspection window and the closing door of the elongated opening.

17. The device of claim 1, wherein the retaining members comprise:
a support element arranged at a first end portion of the elongated seat; and
a pushing element that, when the device is in the initial configuration, is arranged at a second end portion of the elongated seat, the pushing element being movable towards the support element to transition the device from the initial configuration to the final configuration.

18. The device according to claim 17, wherein the container comprises:
an end portion proximate to the support element; and
the support element comprises an abutment surface that, when the device is in the initial configuration, is in a position distal from the end portion and that, when the device is in the final configuration, is in abutment against the end portion.

19. The device of claim 17, wherein:
the cartridge comprises a plunger sealingly arranged inside the container; and
the pushing element comprises a piston configured to exert a thrust on the plunger, wherein the thrust of the piston on the plunger is configured to cause a displacement of the cartridge towards the support element before the device reaches the final configuration, and wherein the thrust of the piston on the plunger is configured to cause a sliding of the plunger inside the container and an outflow of the medicament from the container through a piercing needle after the device reaches the final configuration.

20. The device of claim 9, wherein the closing door comprises, on a surface of the closing door facing the elongated seat, a perimeter rib protruding towards the elongated seat and having a shape complementary to a profile of the cartridge.
